(19) United States
(12) Patent Application Publication
(10) Pub. No.: US 2024/0123009 A1
(43) Pub. Date: Apr. 18, 2024

(54) LOW-PRESSURE INFUSION OF BOTANICALS WITH VOLATILE AEROMATIC AND FLAVOR COMPONETNS INCLUDING TERPENES, ALDEHYDES, KETONES AND ESTERS

(71) Applicants: Ryan Day, Birmingham, MI (US); Samantha Bevins, Canton, MI (US)

(72) Inventors: Ryan Day, Birmingham, MI (US); Samantha Bevins, Canton, MI (US)

(21) Appl. No.: 18/447,023

(22) Filed: Aug. 9, 2023

(51) Int. Cl. A61K 36/185; A24B 15/30; A24D 1/00; A24D 1/02; A24D 1/18

(52) U.S. Cl. CPC A61K 36/185; A24B 15/303; A24D 1/002; A24D 1/02; A24D 1/18; A61K 2236/17

(57) ABSTRACT

The present disclosure relates to an infused botanical such as cannabis flowers that contain volatile and aromatic components, such as cannabinoids, terpenes, aldehydes, ketones and esters. A process of infusing these botanicals with terpenes, aldehydes, ketones and esters helps to improve flavor profile, aroma, and enhance bio-efficacy. The total terpene content of the infused botanical is between 1% to 15% w/w of the one or more botanicals in one embodiment, and between 2.5 to 3.5% w/w in another embodiment. These terpene content percentages fortify the infused botanical with higher terpene numbers that that which occurs naturally in one embodiment of the invention. In another embodiment, the invention restores or bolsters an initial terpene, aldehydes, ketone and ester profile. This invention can be used to infuse pre-rolled botanical cigarettes to extend shelf life and aroma, as well as to infuse rolling papers or cellulosic packages for botanicals and other products.

### FIELD OF THE INVENTION

The present invention relates to the field of supplementing botanicals with volatile organic materials such as plant-derived terpenes, and more particularly to infusing cannabis with terpenes, aldehydes, ketones, and esters to improve shelf-life, aroma, taste and bio-activity.

### BACKGROUND OF THE INVENTION

This background describes the technical background, discusses problems encountered in the technical field, and is not to be construed as prior art.

Cannabis is a versatile plant known for its therapeutic compounds, contains a myriad of valuable bio-active constituents, including terpenes.

A terpene is a volatile aromatic compound responsible for the fragrance is and flavor characteristics in many plants. Many terpenes have been identified and found in abundance in the biomass, and particular the flowers of cannabis. Terpenes can modulate the effects of cannabinoids. Cannabinoids directly impact the cannabinoid receptors in humans. They can agonize, or anatagonize, cannabinoid receptors in vivo, for example. Many terpenes can provide supplemental effects to these receptors when combination with particular cannabinoids, and sometimes independently. Other flavorful and aromatic components of cannabis and many other plants include aldehydes, ketones, and esters.

Growing conditions and genetics both directly influence the final terpene concentration in plants, which dictates the intensity and variety of their aromas and flavors. Among several thousands of cannabis strains, some are available with a larger cannabinoid and terpene concentration than others. The highest concentrations of terpenes in the cannabis plant is typically found in the mature flowers or buds.

Harvesting conditions and growth parameters also massively influence the buds' aroma and flavor. The harvesting conditions and growth parameters include the right amount of nutrients provided to the plant, lightening conditions, and temperature conditions.

High temperatures or drastic temperature variations can cause terpene loss in cannabis plants, whereas excess fertilizers can deteriorate the aroma and flavor of cannabis flowers. Sudden changes in humidity, rain or frost, and insect infestation can also cause a decrease in terpene concentration. In adverse instances, harvest ends up losing a majority of terpenes in the final product if buds are dried too fast or in the presence of light. Hence, cannabis may loose bioactive aromas, and its overall effects if not cared for sufficiently. Aroma, and terpenes, may degrade over time during storage. Dried cannabis flowers have a limited shelf life, after which, quality aroma, bio-efficacy, and value degrades.

Volatile aromatic compounds such as terpenes can be added to the flower buds to increase the aroma, flavor and thus shelf life. Several methods have been used to add cannabinoids to botanicals like Cannabis sativa l., including direct spraying, soaking and pressing isolated cannabinoids on the dried cannabis flowers.

Terpenes have also been added to amplify desirable effects of particular cannabinoids, to improve aroma and flavor, and to extend shelf life. Terpenes have been sprayed on cannabis flowers, and such flowers can be soaked in particular terpene baths.

In direct spraying, diluted terpenes are sprayed evenly onto cannabis buds or extracts. After absorption, the material undergoes drying and curing to achieve desired results, such as smokability. An exemplary spraying method is discussed.

Direct Spraying Method Example:
a. Preparation: Start by selecting the desired terpene or terpene blend that complements the characteristics of the cannabis material.
b. Dilution: Dilute the terpenes to an appropriate concentration to ensure controlled and even application. This may involve diluting with a food-grade solvent or carrier oil.
c. Spraying Process: Place the cannabis material, such as buds or extracted concentrates, in a suitable container or tray. Distribute the diluted terpene solution is over the cannabis material evenly using a fine mist sprayer to ensure thorough coverage.
d. Absorption and Drying: Allow the sprayed cannabis material to absorb the terpenes for a designated period, ensuring proper airflow to facilitate evaporation of the solvent or carrier oil. This allows the terpenes to adhere to the surface of the cannabis material.
e. Curing: After the drying process, it is recommended to cure the sprayed cannabis material to allow the terpenes to integrate and enhance the flavor and aroma profile fully.

An exemplary soaking technique involves immersing the cannabis material in a container filled with a diluted terpene solution. After soaking for a defined period, the material is drained and dried, followed by a curing process to integrate the terpenes.

Soaking Method:
a. Preparation: Similar to the direct spraying method, select the desired terpenes and dilute them to an appropriate concentration.
b. Container Selection: Choose a container that can accommodate the cannabis material and the terpene solution while allowing for thorough immersion.
c. Immersion Process: Place the cannabis material in the container and pour the diluted terpene solution. Ensure that the cannabis is fully submerged and let it soak for a defined period, allowing the terpenes to infuse into the material.
d. Drainage and Drying: After immersing for a defined period, remove the cannabis material from the terpene solution and allow it to drain excess liquid. Place is the material on a tray or drying rack to dry, ensuring adequate airflow to facilitate the evaporation of any remaining solvent or carrier oil.
e. Curing: Similar to the direct spraying method, consider curing the soaked cannabis material to allow the terpenes to integrate and develop the desired flavor and aroma characteristics.

Combining Spraying and Soaking Methods: In some cases, combining spraying and soaking methods leads to more pronounced results. This involves spraying the cannabis material initially and subsequently soaking it in a terpene solution to enhance the aromatic profile further. The duration and sequence of spraying and soaking can be tailored to achieve the desired flavor and aroma intensity.

One drawback of spraying techniques is that the distribution uniformity of aromatic compounds is primarily limited to the surface. When the biomass is crushed, ground, or otherwise parsed for smoking, a few portions exhibit the added compounds, while others do not.

With respect to soaking there is lack of precise control over the volume of added material on a percentage basis (weight by weight) because the amount absorbed depends on numerous factors which may not be readily controlled.

Therefore, there is a need for an improved process of achieving a specifically curated profile of volatile aromatic compounds in a botanical such as the flowers of cannabis, and that can precisely infuse cannabis biomass with a desired terpene profile.

### SUMMARY OF THE INVENTION

An object of the present invention is to provide an infused botanical with additional terpenes, aldehydes, ketones, and esters phenyls, alcohols, carboxylic acid, and a processes for producing the same to improve profile, aroma, extend shelf life, enhance bio-efficacy, and boost flavor when smoked or processed into a concentrated product.

According to one aspect of the invention, an infused botanical product results. Although cannabis flowers are the primary focus of this patent application, the invention can also be used to infuse any smokable biomass including tobacco, mint, catnip, or other smokable herbage. While terpene infusion is discussed by way of example the infusion process can include infusion of aldehydes, ketones, esters phenyls, alcohols, and carboxylic acids to achieve a desired flavor and aroma profile of an infused botanical.

The infused botanical comprises one or more botanicals containing cannabinoids or terpenes, or both.

In one embodiment, dried botanicals are provided and certain desired terpenes are removed, and certain desired terpenes are infused, yielding a desired terpene profile. In a variation of this embodiment, the removal of terpenes (through degradation or volatilization) occurs naturally during storage of botanical biomass. Thus the terpene profile changes over time, as do the profiles of other aromatic compounds including aldehydes, ketones, and esters phenyls, alcohols, and carboxylic acids.

In one embodiment, this invention restores the original terpene profile, or restores and augments the original terpene profile of dried botanicals such as cannabis. This is especially beneficial for smokable cannabis. An advantage of the is methods of the present invention, is that no additives are required that do not originate from the cannabis. Another advantage is that the original terpene profile of a particular strain can be re-constituted to extend shelf life.

Where botanicals have an initial terpene profile, the one or more terpenes infused in the one or more botanicals change the initial terpene profile by increasing terpene concentrations of selected terpenes. By increasing the terpene concentrations of selected terpenes, the cannabis flower can be smoked to achieve improved sleep, improved appetite, improved energy levels, improved pain management, or other desirable effect. Similar effects can be achieved by optimal terpene profiles in extracted oils, wax, shatter, and edible product containing such compounds.

In certain cases where a batch of botanicals is separated into selected flowers, and other vegetative biomass, the vegetative biomass including leaves can undergo an extraction and terpene isolation process. An example of processing can include CO₂ extraction and High Performance Liquid Chromatography (HPLC) isolation of discrete terpenes. The isolated terpenes can be selectively infused into the dried botanical flowers, for example.

Using the native terpenes from non-flowering biomass of a dried botanical improves manufacturing efficiency of the fortified flower products, and also creates an additive and adulterant free dried botanical product for smoking or further processing. Such use also increases the flavors and aromas for those containing less than optimal native terpene profile.

In an embodiment, the dried botanicals contain at least one or more terpenes selected from β-caryophyllene, bisabolol, carene, eucalyptol (cineole), humulene, limeonene, linalool, myrcene, neroidol, β-ocimene, pinene, terpineol, terpinolene or a combination thereof. Such terpenes can be present primarily in the flower of such dried botanicals, and at least some in lesser concentrations in the leaves or other biomass.

In another embodiment, the one or more terpenes are selected from the group of terpenes, naturally occurring in the one or more botanicals containing cannabinoids and terpenes and used for infusion into smokable flowers, or orally consumable products.

In another embodiment, the one or more terpenes are extracted or infused from the at least one portion of the one or more botanicals under vacuum at a negative pressure (below atmospheric pressure) ranging from 3160000 μm to 1600 μm.

In one embodiment, the pressure is reduced linearly to 16000 μm. In another embodiment, the pressure is reduced incrementally and maintained at each increment for a pre-determined period until 16000 μm is reached or passed. In another embodiment, the pressure is varied in a sinusoidal manner to infuse various terpenes simultaneously and consistently, where more than one sinusoidal variation reduces pressure to at or below 16000 μm.

According to another aspect, the present invention provides an infused cannabis flower. The Infused cannabis flower comprises dried cannabis flower biomass having naturally occurring cannabinoids and at least one or more naturally occurring terpene. The dried cannabis flower being infused with additional terpenes. The dried cannabis flower biomass has naturally occurring cannabinoids, including acid forms of tetrahydrocannabinol and cannabidiol. The one or more naturally occurring terpene can be selected from β-caryophyllene, bisabolol, carene, eucalyptol (cineole), humulene, limeonene, linalool, myrcene, neroidol, β-ocimene, is pinene, terpineol, terpinolene or a combination thereof. The dried cannabis flower being infused with additional terpenes, including at least one or more terpene selected from β-caryophyllene, bisabolol, carene, eucalyptol (cineole), humulene, limeonene, linalool, myrcene, neroidol, β-ocimene, pinene, terpineol, terpinolene or a combination thereof.

The process of infusion occurs at low temperature and pressure within the ranges of 3160000 μm to 16000 μm to inhibit volatilization of the naturally occurring terpenes and to inhibit decarboxylation of the naturally occurring cannabinoids. Low temperature and pressure conditions play a critical role in keeping the naturally occurring terpenes intact and avoiding any degradation of the cannabinoids present.

In an embodiment, the naturally occurring terpenes define an initial terpene profile, which degrades at room temperature over time. The additional terpenes, thus, cause the infused cannabis flower to regain the initial terpene profile.

In an alternative embodiment, the naturally occurring terpenes define an initial terpene profile, which over time degrades at room temperature; the additional terpenes cause the infused cannabis flower to exceed the initial terpene profile by increasing the concentration in the infused cannabis flower with a low-pressure infusion process.

According to another aspect, the present invention provides a process for infusing botanicals with terpenes. The process comprises the steps of a) introducing one or more terpenes in a first compartment; b) introducing one or more botanicals in a second compartment that is in fluid communication with the first compartment; c) heating the first compartment under vacuum to vaporize the one or more terpenes; d) allowing the one or more terpene vapors to penetrate the one or more botanicals at a pre-determined temperature under vacuum; and e) collecting the infused botanicals.

In an embodiment, the amount of the one or more terpenes is 1% to 15% w/w of the botanicals. In a preferred embodiment, the amount of the one or more terpenes is 3.5% w/w of the botanicals.

In another embodiment, the one or more botanicals are selected from one or more cannabis species, including Cannabis sativa, Cannabis indica, Cannabis ruderalis, or a combination thereof. In a preferred embodiment, the one or more botanicals are a cannabis species.

In an embodiment, the one or more botanicals contain any part of the plant of cannabis species, including leaf, root, stein, flower, or a combination thereof.

In an embodiment, the first compartment is heated under vacuum at a negative pressure ranging from 3160000 μm to 16000 μm.

In yet another embodiment, the first compartment is heated under vacuum at a temperature ranging from 20° C.-55° C.

In another embodiment, the time for allowing the terpene vapors to penetrate the botanicals is at least 1.5 hours.

According to another aspect, the present invention provides a process for infusing botanicals with terpenes. The process comprises the steps of a) providing one or more terpenes in a first compartment; b) providing one or more botanicals in a second compartment that is in fluid communication with the first compartment; c) heating the first compartment under vacuum to vaporize the one or more terpenes; d) allowing the one or more terpene vapors to penetrate the one or more botanicals at a pre-determined temperature under vacuum; e) collecting the infused botanicals; f) recovering unused terpenes from the first and second compartments via a vapor trap; and g) repeating the steps from a) to f) for infusing the one or more botanicals with the one or more terpenes in multiple batch processing; wherein the one or more terpenes are the recovered terpenes, fresh terpenes, or a combination thereof.

According to another aspect of the present invention, the present invention provides a process for infusing a dried cannabis flower having an initial terpene profile. The process comprises the steps of a) providing dried cannabis flower having less than 20% moisture content; b) providing the dried cannabis flower in a container; c) reducing the pressure in the container ranging from 3160000 μm to 16000 μm and adding selected terpenes chosen from the group consisting of β-caryophyllene, bisabolol, carene, eucalyptol (cineole), humulene, limeonene, linalool, myrcene, neroidol, β-ocimene, pinene, terpineol, terpinolene and combinations thereof; and d) the added terpenes penetrate the dried cannabis flower to create an infused cannabis product.

In an embodiment, the process further comprises identifying an ideal terpene profile, and the step of infusing the selected terpenes causes the dried cannabis flower to achieve the ideal terpene profile. This can be applied to botanical biomass, pre-rolled cigarettes, rolling paper, or packaging for any product including non-cannabis and non-botanical goods. Such packaging would be absorbent. Examples of absorbent packaging include cellulosic material including paper. Packaging includes containers, bags, clothing tags, and even plastics having an absorbent patch adhered thereto.

Accordingly, the process allows terpenes to rapidly volatilize and turn is into a “fog” at a lower temperature than the normal atmosphere. The process utilizes less terpene than other available methods and enables the use of the same terpenes in multiple batches. The gaseous state of the volatilized terpenes allows them to penetrate the flower without leaving a noticeable sticky or greasy residue. Furthermore, since the first and second compartments are under vacuum, there are no or minimal chances of adding/removing moisture or humidity. The moisture content lost in the initial heating process is reabsorbed once the chamber cools prior to reintroducing the atmosphere. Therefore, vapor infusion under vacuum or low-pressure conditions is a suitable method where the chemical components are too delicate and easily denatured by the high heat used in steam distillation.

### BRIEF DESCRIPTION OF THE DRAWINGS

Various aspects, as well as embodiments of the present invention, are better understood by referring to the following detailed description. To better understand the invention, the detailed description should be read in conjunction with the drawings.

FIG. 1 illustrates a process of infusing botanicals with terpenes in accordance with an embodiment of the present invention;

FIG. 2 illustrates a process of infusing botanicals with terpenes in accordance with another embodiment of the present invention;

FIG. 3 illustrates a process of infusing a dried cannabis flower having an initial terpene profile in accordance with another embodiment of the present invention;

FIG. 4 illustrates an apparatus for infusing botanicals with terpenes in accordance with an embodiment of the present invention;

FIG. 5A illustrates a hemispherical reducer lid in accordance with an embodiment of the present invention;

FIG. 5B illustrates a container in accordance with an embodiment of the present invention;

FIG. 5C illustrates a tri-clamp filter plate in accordance with an embodiment of the present invention;

FIG. 5D illustrates a terpene compartment in accordance with an embodiment of the present invention;

FIG. 5E illustrates a compression ring in accordance with an embodiment of the present invention;

FIG. 5F illustrates high-pressure tri-claps in accordance with an embodiment of the present invention;

FIG. 6 illustrates a block diagram of an apparatus for infusing botanicals with terpene in accordance with an embodiment of the present invention; and

FIG. 7 illustrates a block diagram of multiple container apparatus for infusing botanicals with terpene in accordance with an embodiment of the present invention.

### DETAILED DESCRIPTION OF THE INVENTION

While reading this section (an exemplary embodiment, which describes the exemplary embodiment of the best mode of the invention, hereinafter referred to as “exemplary embodiment”), one should consider the exemplary embodiment as the best mode for practicing the invention during filing of the patent in accordance with the inventor's belief. As a person with ordinary skills in the art may recognize substantially equivalent structures or substantially equivalent acts to achieve the same results in the same manner, or in a dissimilar manner, the exemplary embodiment should not be interpreted as limiting the invention to one embodiment.

The discussion of a species (or a specific item) invokes the genus (the class of items) to which the species belongs, as well as related species in this genus. Similarly, the recitation of a genus invokes the species known in the art. Furthermore, as technology develops, numerous additional alternatives may arise to achieve an aspect of the invention. Such advances are incorporated within their respective genus and should be recognized as being functionally equivalent or structurally equivalent to the aspect shown or described.

Unless otherwise explicitly stated, a function or a term should be interpreted as incorporating all modes of performing the function or defining a term. For instance, the term “vacuum” means “low pressure”, “negative pressure”, “low vapor pressure”, or other similar terms known to a person skilled in the art. Similarly, the terms “terpenes”, “terpene”, or “terpenoids” can be used alternatively.

Cannabis is a herb with a long history of use as fiber, food, oil, and medicine globally. Depending upon the purposes of utilization, it is known by different names; for example, “hemp” when used as a fiber or textile crop and as “marijuana” while it is available for recreational purposes. Among the cannabinoid constituents of cannabis, tetrahydrocannabinol (THC), which is naturally present in the form of an acid (tetrahydrocannabinolic acid, THCA), constitutes the plant's main psychoactive component. Another cannabinoid, cannabidiol (CBD), is active as an antiepileptic agent. Tetrahydrocannabivarin (THCV), cannabinol (CBN), cannabigerol (CBG), and cannabichromene (CBC) are four other primary is cannabinoids found in cannabis. Several other cannabinoids (also known as phytocannabinoids) have been identified and reported to exhibit therapeutic effects. Some of these therapeutic effects include euphoric effects (THC and THCV), analgesic effects (THC, CBD, and THCV), sedative effects (CBD), antipsychotic effects (CBD), anti-inflammatory effects (THC, CBD, CBC, CBG, and CBN), anti-convulsant effects (CBD and CBN), anti-biotic effects (CBC, CBN, and CBG), and anti-fungal effects (CBC and CBG). The non-cannabinoid constituents include cannabinoid phenols, flavonoids, terpenes, alkaloids, and others.

Understanding terpenes: Terpenes are organic compounds found in various plants, including cannabis. They are particularly responsible for the distinctive aromas and flavors exhibited by different cannabis strains. Additionally, some terpenes are believed to contribute to the entourage effect, wherein they enhance the effect of cannabinoids as they synergize the feeling of relaxation, stress relief, energy boost, and maintaining focus along with their underlying pharmaceutical functions.

Some common terpenes found in cannabis and their therapeutic effects are given in Table 1 below:

TABLE 1

| | Dominant Receptor | |
|---|---|---|
| Terpene | Site/s | Potential effects |
| β-Caryophyllene | It binds to | It affects cellular and metabolic systems |
| | cannabinoid-2 | demonstrating antioxidant, anti- |
| | receptors (CB2). | inflammatory, improved wound healing, |
| | | neuroprotective, anti-cancer and |
| | | analgesic properties. |
| Bisabolol | It inhibits | It helps in wound healing, |
| | phospholipase A, | gastroprotection, has antitumor, |
| | cyclooxygenase, and | antioxidant, analgesic and anti- |
| | lipoxygenase | inflammatory properties. |
| | pathways. It stimulates | |
| | gastrointestinal tract | |
| | receptors, thus causing | |
| | smooth muscle | |
| | relaxation. | |
| Carene | It binds to the BZD | It has anti- inflammatory, antimicrobial, |
| | site of α1 and Υ2 | and anxiolytic benefits and stimulates |
| | subunits of GABAA- | memory retention. It shows a sleep- |
| | BZD receptor. | enhancing effect by acting as a positive |
| | | modulator for GABAA- BZD receptor. |
| Eucalyptol | It binds to PPARγ. | It shows anti-inflammatory effects, |
| (Cineole) | | indicating its possible use in treating |
| | | IBD. |
| Humulene | It binds to CB1R. | It shows strong anti-inflammatory |
| | | properties comparable to |
| | | dexamethasone systemically and |
| | | topically, as well as analgesic |
| | | properties. It causes weight loss and |
| | | appetite suppression. It is also said to |
| | | have anti-bacterial properties. |
| Limeonene | It binds to serotonin | It shows anti-inflammatory, gastro- |
| | receptor, 5- HT1A | protective, anti-nociceptive, anti-tumor, |
| | leading to increased | and neuroprotective activity. It causes |
| | serotonin in the | mood enhancement and stress reduction |
| | prefrontal cortex and | as well as sedation and immune- |
| | dopamine in the | stimulation. |
| | hippocampus. | |
| Linalool | It binds to glutamate | It shows sedative, anxiolytic and |
| | and GABA | anticonvulsant effects. It is used in |
| | neurotransmitter | treatment of anxiety, sedation and |
| | systems. | relaxation, pain, arthritis, depression, |
| | | seizures and insomnia. |
| Myrcene | It binds to alpha 2- | It is an antioxidant as well as useful in |
| | adrenoreceptorsalpha | reducing inflammation, anxiety and |
| | 2-adrenoreceptors. | chronic pain. It is a recognized sedative, |
| | | potentiating sleep at high doses and is |
| | | employed as a sleep aid in Germany. |
| Neroidol | It binds to the | It has sedative properties and is useful in |
| | GABAergic system | insomnia as well as for pain. |
| | which is responsible | |
| | for its analgesic | |
| | activity. | |
| β-ocimene | It alters plasma | It shows anticonvulsant activity, |
| | membrane | antifungal activity, antitumor activity. |
| | permeability and | |
| | interacts with proteins | |
| | and lipids. | |
| Pinene | It acts on multiple | It has sedative, hypnotic, and anxiolytic |
| | neurologic pathways, | properties along with significant anti- |
| | including GABAergic, | inflammatory and analgesic benefits. It |
| | cholinergic, | also plays an insect-repellent role in |
| | dopaminergic, | plants and is a bronchodilator in humans |
| | serotoninergic, | at low exposure levels. It improves a |
| | adrenergic, | person's ability to focus their attention |
| | noradrenergic | as well as to reduce perception of stress. |
| | neurotransmitter | |
| | systems. | |
| Terpineol | It decreases leucocyte | It shows anti-inflammatory, analgesic |
| | migration and TNF | and antioxidant properties and it may |
| | levels and suppresses | have anxiolytic and sedative effects. It |
| | the production of | has bactericidal and antifungal |
| | inflammatory | properties and strong anti-proliferative |
| | mediators (e.g., NF- | activity on cancer. It has also been |
| | κB, p38, ERK, and | investigated for the treatment of allergic |
| | MAPK signaling | inflammation and asthma. |
| | pathways) in human | |
| | macrophages. | |
| Terpinolene | It acts upon 5HT1A | It shows sedating and calming effects. It |
| | receptors, reduction of | may have properties that are sedating, |
| | IL-1β and TNF-α | anxiolytic, anti-cancer, anti-bacterial, |
| | cytokines. | and anti-oxidant, and may help prevent |
| | | heart disease. |

In cannabis industry, there is a huge demand of adding either cannabis terpenes or botanically-derived terpenes to consumable oils and other edibles that get depleted either at crop level or after harvesting and storage. By adding these volatile organic compounds, cannabis enthusiasts and manufacturers can customize the sensory experience of cannabis products by incorporating the flavors and aromas.

It is essential to note that when working with volatile aromatic compounds, proper safety precautions should be followed. This includes working in a well-ventilated area, using appropriate personal protective equipment, and adhering to the recommended dilution ratios to ensure safe and controlled application. Numerous terpene recovery techniques have been developed so far using many extraction methods, including distillation. Extracting terpenes from cannabis biomass requires careful consideration and specialized techniques. Although most of the constituents remain intact during distillation, a few monoterpenes may undergo chemical changes or are often lost due to the nature of the distillation process.

Commonly available terpenes with their boiling points are listed in TABLE 2 below:

TABLE 2

| Terpene | Boiling point |
|---|---|
| β-Caryophyllene | 473.5° F. - (245.27778° C.) |
| Bisabolol | 599.00° F. - (315.00° C.) |
| Carene | 388° F. - (170-172° C.) |
| Eucalyptol (Cineole) | 348.8° F. - (176-177° C.) |
| Humulene | 225° F. - (107° C.) |
| Limeonene | 350° F. - (177° C.) |
| Linalool | 388.4° F. - (198° C.) |
| Myrcene | roughly 330° F. - (166° C. and 168° C.) |
| Neroidol | 251.6° F. - (122° C.) |
| β-ocimene | 345.2° F. - (174.00 to 175.00° C.) |
| Pinene | 312° F. - (156° C.) |
| Terpineol | 426.2° F. - (219° C.) |
| Terpinolene | 426.2° F. - (363.2° C.) |

The Role of Low Pressure: Low-pressure extraction techniques offer a gentle and efficient way to obtain terpenes from cannabis biomass. Unlike high-pressure methods that may risk damaging the delicate terpene molecules, low-pressure extraction provides a controlled environment that preserves the integrity of these valuable compounds. By carefully adjusting the pressure, it is possible to isolate and collect terpenes while minimizing the risk of thermal degradation. Pressures can be lowered in a linear progression, stepped down, or varied along a curve engineered to volatilize particular terpenes over a known time period.

In an exemplary embodiment, the current invention provides a device for holding extracted terpenes in a pressure chamber and infusing a botanical such as dried cannabis flower. The invention, can achieve a desired terpene profile in the infused dried cannabis flower.

The botanical is cannabis biomass and any strain or variant thereof, including Cannabis sativa l., Cannabis indica, Cannabis ruderalis. Preferably, the botanical is a strain of Cannabis sativa l. species.

The cannabis biomass includes at least one of flowers, leaves, hurd, seeds, roots, peel, or a combination thereof. Generally, cannabinoids are found in all the plant parts of Cannabis sativa l. with the exception of the seeds. The floral bracts and fruit stalks contain the highest cannabinoid concentrations.

Alternatively, the botanical for infusion may be a strain of Amaranthus dubius, Arctostaphylos uva-ursi, Argemone mexicana, Arnica, Artemisia vulgaris, Calea zacatechichi, Canavalia maritima, Cecropia mexicana, Cestrum noctumum, Cynoglossum virginianum, Cytisus scoparius, Entada rheedii, Eschscholzia californica, Fittonia albivenis, Hippobroma longiflora, Humulus japonica, Humulus lupulus, Lactuca virosa, Laggera alata, Leonotis leonurus, Leonurus cardiaca, Leonurus sibiricus, Lobelia cardinalis, Lobelia inflata, Lobelia siphilitica, Nepeta cataria, nicotiana (i.e., tobacco), Nymphaea alba, Opium poppy, Passiflora incarnata, Pedicularis densiflora, Pedicularis groenlandica, Salvia divinorum, Salvia dorrii, Salvia, Scutellaria galericulata, Scutellaria lateriflora, Scutellaria nana, Scutellaria, Sida acuta, Sida rhombifolia, Silene capensis, Syzygium aromaticum, Tagetes lucida, Tarchonanthus camphoratus, Turnera diffusa, Verbascum, and Zornia latifolia, or a combination thereof.

The terpenes are selected from the group consisting of, but not limited to bisabolol, carene, eucalyptol (cineole), humulene, limeonene, linalool, myrcene, β-ocimene, pinene, terpineol, terpinolene, pulegone, p-cymene, borneol, β-caryophyllene, caryophyllene oxide, nerolidol, phytol, eugenol, sabinene, linalyl acetate, camphor, chamazulene, beta-farnesene, alpha-humulene, benzyl benzoate, benzyl acetate, geraniol, geranyl acetate, gamma-terpinene, and a combination thereof.

An optimized process for the infusion of botanicals with terpenes at low pressure is provided. This process avoids any degradation products e.g. those produced during decarboxylation of the naturally occurring cannabinoids due to high-temperature conditions being used to vaporize the terpenes traditionally. Also, by following this process, the original content of terpenes remains intact since volatilization of the naturally occurring terpenes is inhibited. The cannabis enthusiasts and product manufacturers can customize and elevate the sensory experience of cannabis products by harnessing the power of volatile aromatic compounds, such as terpenes. The product can be stored and utilized as per demand. If the terpene content gets depleted after a long period of storage, it can again be restored by following the above process and put to use. Thus, dried cannabis flower, shake, or smokable biomass need not be discarded or discounted reduction in aromas, flavors, or medicinal value.

Many terpenes express synergistic effects, when combined with particular cannabinoids. This is known as the entourage effect in general. Particular effects on cannabinoid receptors, illnesses, and experience of a user can be engineered.

The present invention provides an infused cannabis flower. The infused cannabis flower comprises dried cannabis flower, at least one or more naturally occurring terpene. The dried flower biomass has naturally occurring cannabinoids including acid forms of tetrahydrocannabinol and cannabidiol. The at least one or more naturally occurring terpene selected from β-caryophyllene, bisabolol, carene, eucalyptol (cineole), humulene, limeonene, linalool, myrcene, neroidol, β-ocimene, pinene, terpineol, terpinolene or a combination thereof. The dried cannabis flower is infused with additional terpenes including at least one or more terpene selected from β-caryophyllene, bisabolol, carene, eucalyptol (cineole), humulene, limeonene, linalool, myrcene, neroidol, β-ocimene, pinene, terpineol, terpinolene or a combination thereof.

The process of infusion occurs at low temperature and pressure within the ranges of 3160000 μm to 16000 μm to inhibit volatilization of the naturally occurring terpenes, and to inhibit decarboxylation of the naturally occurring cannabinoids.

Naturally occurring terpenes in the botanicals, preferably a cannabis is flower, define an initial terpene profile, which degrades at room temperature with time, the additional terpenes cause the infused cannabis flower to regain the initial terpene profile. Alternatively, the additional terpenes cause the infused cannabis flower to exceed the initial terpene profile by increasing the concentration in the infused cannabis flower with a low-pressure infusion process.

Profiles of naturally occurring, and volatile, aromatic and flavor components including terpenes, aldehydes, ketones, and esters, phenyls, alcohols, and carboxylic acids also define their respective profiles, and these degrade over time naturally, or when exposed to heat, light, and other derogatory conditions. Collectively these are termed “volatile components”.

The present invention can bolster the profiles of any of these volatile components, or restore the naturally occurring amount of these volatile components. Thus, while the present patent application describes primarily terpenes and infusion of the same, the invention further encompasses other volatile components, including aldehydes, ketones, and esters phenyls, alcohols, and carboxylic acids in order to restore or bolster a “fresh” smelling and flavorful botanical, such as Cannabis sativa flower.

In an embodiment of the invention a dual chamber device is provided. In the first chamber at least one isolated terpene, aldehyde, ketone, and ester, phenyl, alcohol, and carboxylic acid is presented on a splatter platter. The temperature is maintained in this first chamber at a maximum of 90° F. to inhibit degradation.

In another embodiment of the invention, multiple first chambers can be provided, each having a temperature not exceeding 90° F. Such multiple first chambers each having a splatter platter for holding a specific isolated terpene, aldehyde, ketone, and ester, phenyl, alcohol, and carboxylic acid on a respective splatter plater. The multiple first chambers are in fluid communication with a is second chamber, where botanical biomass to be infused is enclosed.

Thermal energy, or heat, naturally flows from hot toward cold. Thus any temperature differential from the heated first chamber having the splatter platter to the second chamber having relatively cooler biomass, will naturally flow the heated terpenes and other components from the first chamber to the second chamber.

The top of the vessel, hemispherical dome lid, will remain the coolest part of the machine as the heat source is located at the bottom in one embodiment. Thus a laminar flow generated from temperature differentials cause terpenes and other components to rise through the biomass m the second chamber and thereby infuse the biomass material via the gentile laminar flow. This avoids the use of a pump which may undesirably impart heat to the fluid being pumped.

In one embodiment, the temperature in the first chamber at the splatter platter is 130° F. and the temperature in the second chamber where the biomass sits is 90° F. Thus there is a 40° F. temperature differential to activate and maintain fluid flow.

In another, preferred embodiment, the temperature at the spatter platter is 69° F. and the temperature of the second chamber is maintained below 60° F. to create a minimum of 9° F. temperature differential to activate and maintain fluid flow. Having a minimum of 9° F. may take longer for the infusion process to complete but may also more precisely regulate the amount of terpenes or other volatile components being infused.

In another preferred embodiment, the temperature of the second chamber is cooled to at least 32° F. to reduce any exit of terpenes from the second chamber. Alternatively, a chiller can be attached to re-capture unused terpenes and other volatile components.

Ideally a 37-40° F. temperature differential between the first and second chambers is provided to induce and maintain fluid flow of the volatile components including terpenes. Further, the process is accomplished under a vacuum with constant pressure, stepped pressures to infuse particular volatile components in sequence, or in sinusoidal waves of varying pressures to mix the volatile components and infuse them simultaneously.

The volatile components include Aldehydes. Volatile aldehydes are responsible for the aromas of cannabis arguably just as much as terpenes. An aldehyde functional group consists of carbon with a double bond to an oxygen and a single bond to a hydrogen. Examples of volatile aldehydes found in cannabis include Benzaldehyde, Decanal, Heptanal, Octanal, Hexanal, and

Isobutyraldehyde. Preferably, these aldehydes are isolated from a batch of Cannabis sativa biomass (preferably the leaves) and then infused into the dried cannabis flower of the present invention to achieve an “additive” free cannabis flower product.

The volatile components include Esters. Esters are compounds that are formed by joining together an alcohol and a carboxylic acid with an oxygen atom. These compounds are responsible for giving many fruits and vegetables their characteristic aromas and flavors. Preferably the Esters are cannabis-derived and isolated therefrom. In other aspects of the invention, the Esters are synthesized from carboxylic acids.

The volatile components include Ketones. Ketones are compounds which consist of two different carbon containing compounds are joined together by a double bonded oxygen. Volatile Ketones found in cannabis include 2-methyl-4-heptanone and 2-methyl-3-heptanone. The methylisohexenyl ketone and 1-chloroacetophenone may contribute to the characteristic aroma of cannabis.

Process methods of the present invention are intended to include the above volatile components in addition to the terpenes.

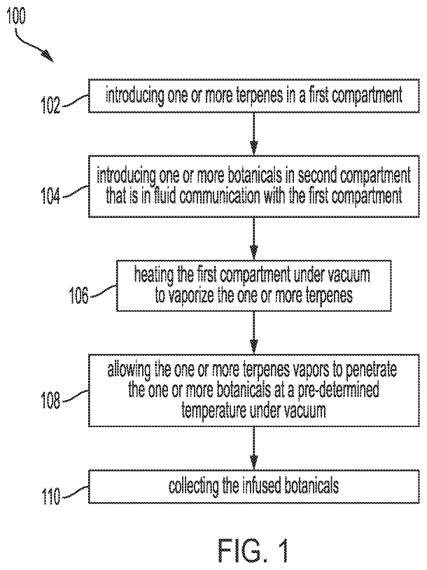

FIG. 1 illustrates a process 100 of infusing botanicals with terpenes in accordance with an embodiment of the present invention comprising the following steps: (a) introducing (102) one or more terpenes in a first compartment; (b) introducing (104) one or more botanicals in second compartment that is in fluid communication with the first compartment; (c) heating (106) the first compartment under vacuum to vaporize the one or more terpenes; (d) allowing (108) the one or more terpenes vapors to penetrate the one or more botanicals at a pre-determined temperature under vacuum; and (e) collecting (110) the infused botanicals. It can e appreciated that the temperature can be changed slowly over time and the pressure can vary to selectively adjust the concentrations of volatilized terpene that pass into the second compartment, and the rate that such volatilized terpenes pass into the second compartment. The first compartment having a higher temperature than the second compartment.

The amount of the one or more terpenes used for infusion can vary from 0.1% to 30% w/w of the one or more botanicals as per desired terpene profile in the end product. Preferably, the amount of the one or more terpenes used for infusion is 1% to 15% w/w of the one or more botanicals. More preferably, the amount of the one or more terpenes used for infusion is 3.5% w/w of the one or more botanicals.

The botanicals are selected from one or more cannabis species, including Cannabis sativa, Cannabis indica, Cannabis ruderalis, or a combination thereof. Preferably, the botanical is a cannabis species. The botanicals contain any part of the plant of cannabis species, including leaf, root, stein, flower, or a combination thereof.

The terpenes are infused or drawn into the low-pressure atmosphere from a terpene compartment. The terpene compartment can have sensors and a control system to detect the particular volume of terpenes. The apparatus automatically gets turned off after a particular time or when the terpene compartment is empty. The time allowed for vapor penetration of terpenes is also crucial. Each terpene may have a different application time. The terpenes are allowed to penetrate the botanicals for a min duration of 1 hour. Preferably, the terpenes are allowed to penetrate the botanicals for a min duration of 1.5 hours.

Source of terpenes: Commercially available terpenes, if used for infusing cannabis, can be considered an adulterant under various state and local regulations due to different moisture content, traces of pesticides, fungicides, or growth enhancers. Therefore, the terpenes used in the present invention can first be extracted or isolated from a portion of target cannabis biomass without limitation, via methods like distillation, particularly under low-pressure conditions. Such extracted terpenes are then infused in the remaining portion to augment the sensory and medicinal effects of the cannabinoids present therein. In distillation, cannabis biomass can include flowers, leaves, wood, bark, roots, peel, and a combination thereof.

The present invention provides an infused botanical with a moisture content of 0.1%-20%, comprising one or more cannabinoids; and one or more terpenes infused in the botanical up to an enhanced terpenoid concentration, wherein the terpene is naturally occurring in the botanical, wherein the terpene is selected from the group consisting of pulegone, terpineol-4-ol, p-cymene, borneol, eugenol, sabinene, linalyl acetate, chamazulene, beta-farnesene, benzyl benzoate, benzyl acetate, geraniol, geranyl acetate or a combination thereof.

Moisture content will remain unchanged as no moisture leaves the apparatus. However, moisture can be adjusted in the infused botanicals by adding that present amount of water to achieve desired moisture level into the terpene compartment and running as usual. Optimal moisture content of the dried or infused botanical is between 9-20%.

The temperature and pressure conditions are very critical for optimizing the process. The temperature at which volatilization happens can vary from 15° C. to 80° C. More preferably, the optimal temperature ranges from 20° C.-55° C. Similarly, the vapor pressure is managed in the range of 4000000 μm (micrometer Hg) to 10000 μm (micrometer Hg). Preferably, the vapor pressure is maintained in the range of 3160000 μm (4.21 Bar) to 16000 μm or (0.02133 Bar). The pressure can be increased linearly and maintained at a consistent level or can be cycled in a sinusoidal manner to optimize penetration. Therefore, using this optimized process of infusing terpenes into dried cannabis biomass, the initial depleted terpene profile can be regained and even enhanced to attain desired pharmacological and sensory effects.

Infusing botanicals with a terpene at a pre-determined temperature under vacuum is optionally followed by recovering unused terpenes. The unused terpenes can be recovered using a vapor trap to condense the terpene vapors.

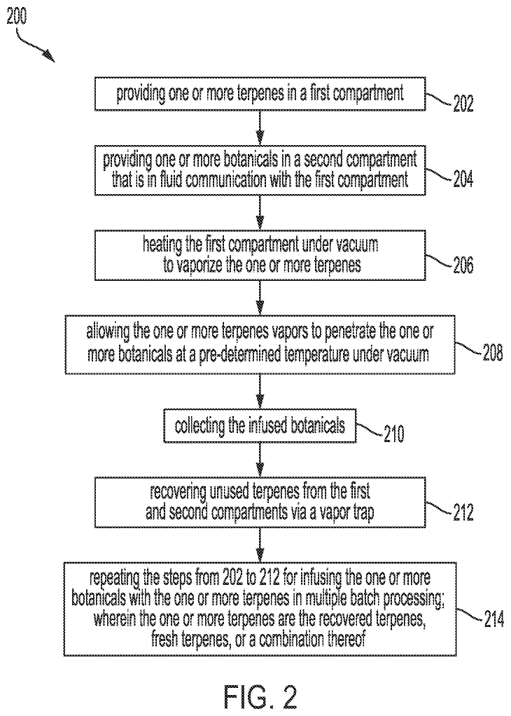

FIG. 2 illustrates a process 200 of infusing botanicals with terpenes in accordance with an embodiment of the present invention comprising the following steps: (a) providing (202) one or more terpenes in a first compartment; (b) providing (204) one or more botanicals in a second compartment that is in fluid communication with the first compartment; (c) heating (206) the first compartment under vacuum to vaporize the one or more terpenes; (d) allowing (208) the one or more terpenes vapors to penetrate the one or more botanicals at a pre-determined temperature under vacuum; (e) collecting (210) the infused botanicals; (f) recovering (212) unused terpenes from the first and second compartments via a is vapor trap; and (g) repeating (214) the steps from a) to f) for infusing the one or more botanicals with the one or more terpenes in multiple batch processing; wherein the one or more terpenes are the recovered terpenes, fresh terpenes, or a combination thereof.

It can be appreciated that numerous compartments can be used with discrete terpenes so that the terpene profile can be specifically managed and so that an operator can observe when all the compartmentalized terpenes are volatilized.

Providing botanicals in second compartment includes providing cannabis biomass comprising at least one cannabinoid, such as a cannabinoid selected from the group consisting of delta-9-tetrahydrocannabinol (THC), cannabidiol (CBD), cannabichromene (CBC), cannabigerol (CBG), tetrahydrocannabivarin (THCV), cannabidivarin (SBDV) and cannabinol (CBN).

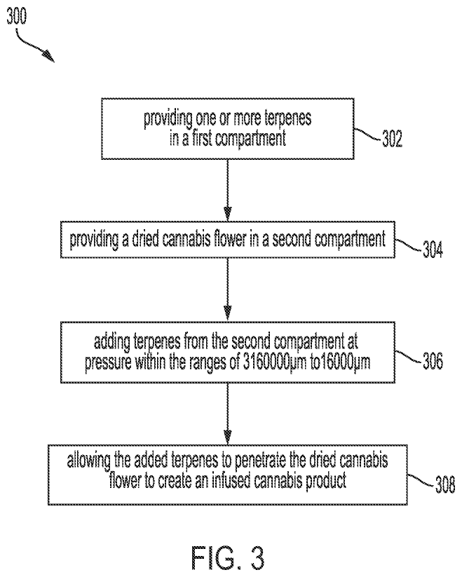

FIG. 3 illustrates a process 300 of infusing a dried cannabis flower having an initial terpene profile in accordance with an embodiment of the present invention comprising the following steps: (a) providing (302) dried cannabis flower having less than 20% moisture content; (b) providing (304) the dried cannabis flower in a container; (c) reducing (306) the pressure in the container, ranging from 3160000 μm to 16000 μm and adding selected terpenes chosen from the group consisting of β-caryophyllene, bisabolol, carene, eucalyptol (cineole), humulene, limeonene, linalool, myrcene, neroidol, β-ocimene, pinene, terpineol, terpinolene and combinations thereof; and (d) allowing (308) the added terpenes to penetrate the dried cannabis flower to create an infused cannabis product. Thus, the terpenes used for infusion are derived from the same strain of botanicals to which they are infused in the next step.

An infused cannabis flower, obtained by infusing dried cannabis flower biomass having less than 20% moisture content, with additional terpenes at low temperature and pressure conditions, is provided. The dried cannabis flower is provided in a container followed by reducing the pressure in the container to 3160000 μm to 16000 μm. The selected terpenes are then added under low-pressure conditions such that they penetrate the dried cannabis flower to create an infused cannabis product. Thus, the initial terpene profile present in dried cannabis flower is tested before infusion, and accordingly, the ideal terpene profile can be achieved by infusing additional terpenes of interest, thereby following the process of infusion.

In an exemplary embodiment, where an entourage effect between a cannabinoid and terpenoids is desired, botanicals other than cannabis may comprise at least one added cannabinoids, such as a cannabinoid selected from the group consisting of delta-9-tetrahydrocannabinol (THC), cannabidiol (CBD), cannabichromene (CBC), cannabigerol (CBG), tetrandrocannabivarin (THCV), cannabidivarin (SBDV) and cannabinol (CBN).

Providing the terpenes in first compartment may include providing one or more terpenes and a solvent, and mixing one or more terpene in the solvent. The solvent can include any liquid, e.g., a volatile liquid, which incorporates a desired amount of the terpene to form a volatile terpene solution.

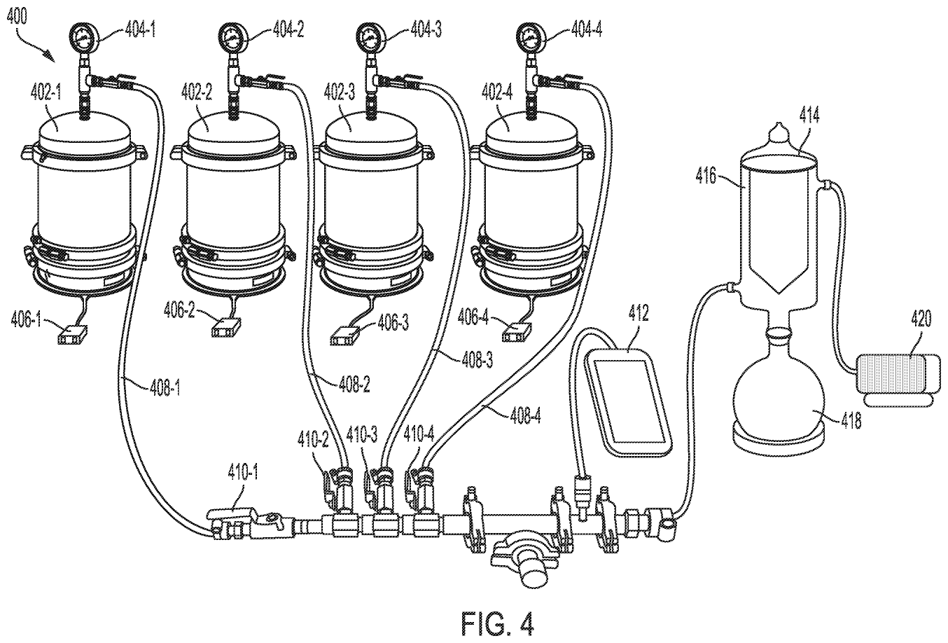

FIG. 4 illustrates an apparatus 400 for infusing botanicals with terpenes in accordance with an embodiment of the present invention. The apparatus 400 for preparing infused botanicals is illustrated while referring to the drawings. The apparatus 400 employs multiple infusion containers simultaneously. It can be operated as a single infusion container mode as well.

The apparatus 400 comprises multiple sealed and insulated containers 402-1, 402-2, 402-3, and 402-4 for carrying the infusion process. Vacuum gauges 404-1, 404-2, 404-3 and 404-4 are installed to measure and maintain vacuum pressure. Digital heating mats 406-1, 406-2, 406-3, 406-4 are also provided in each container to set and control the temperature conditions during the infusion process.

Vacuum hoses 408-1, 408-2, 408-3, and 408-4 corresponding to each container 402-1, 402-2, 402-3, and 402-4 are connected with a digivac 412 for measuring and controlling vacuum via multiple hose ball valves 410-1, 410-2, 410-3, and 410-4. A digivac 412 is further connected with a vacuum pump 420 via a cold vapor trap/distillation flask 414. The cold vapor trap has a first compartment 416, and a second compartment 418, to be used for the recovery of unused terpenes. In another setup, vacuum pump 420 can be directly connected to the digivac 412 instead of the cold vapor trap.

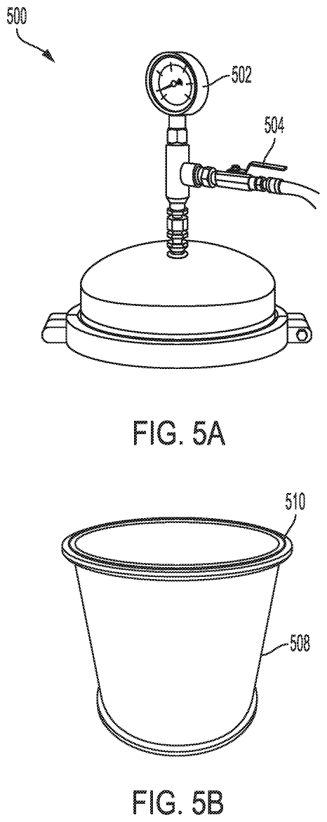

Each container further comprises sub-units, as illustrated by FIGS. 5A to 5B. FIG. 5A illustrates a hemispherical reducer lid 500 in accordance with an embodiment of the present invention. The hemispherical reducer lid 500 is connected to a vacuum gauge 502 and a ball valve 504. FIG. 5B illustrates a stainless-steel tri-clamp spool 508 in accordance with an exemplary embodiment of the present invention. The upper part of the stainless steel tri-clamp spool 508 comprises a nitrile tri-clamp gasket 510. FIG. 5C illustrates a tri-clamp filter plate (stainless steel perforated disk) 512 in accordance with an embodiment of the present invention. The upper part of the tri-clamp filter plate (stainless steel perforated disk) 512 comprises a nitrile tri-clamp gasket 514. FIG. 5D illustrates a weld base spool splatter platter (terpene compartment) 516 in accordance with an embodiment of the present invention. The upper part of the weld base spool splatter platter 516 comprises a nitrile tri-clamp gasket 518. FIG. 5E illustrates a compression ring 520 with 100-micron mesh 522 in accordance with an embodiment of the present invention. FIG. 5F illustrates three high-pressure tri-clamps 524 in accordance with an embodiment of the present invention.

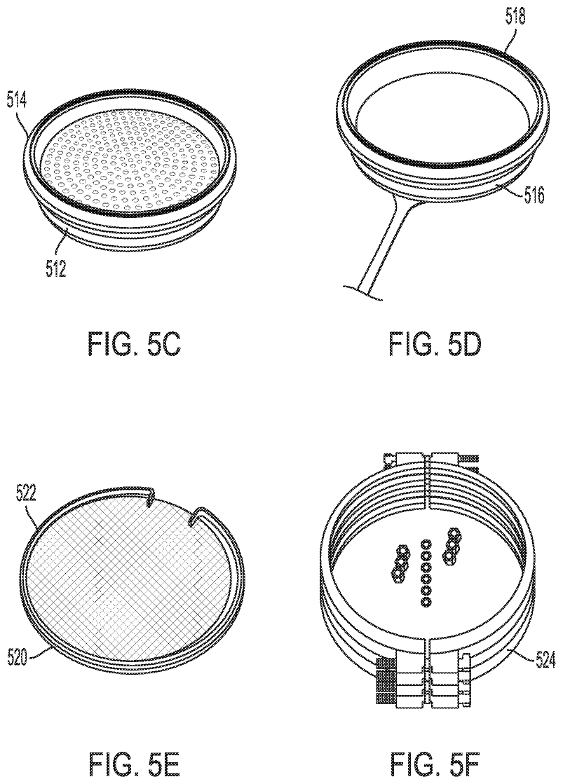

FIG. 6 illustrates a block diagram of apparatus 600 for infusing botanicals with terpene in accordance with an embodiment of the present invention. The apparatus comprises a container 602 having a first compartment 604 and a second compartment 606.

In one embodiment, during operation the first compartment is heated to between 20-55° C. and the second compartment has a lesser temperature than the first compartment so that terpenes in the dried botanical material in the second compartment does not readily volatilize, and so that terpenes from the first compartment more easily condense in and on the dried botanical material.

The container 602 is further connected to a vacuum gauge 608, a vacuum pump 610, a pressure sensor 612, a temperature sensor 614, a heater 616, and a digivac 618.

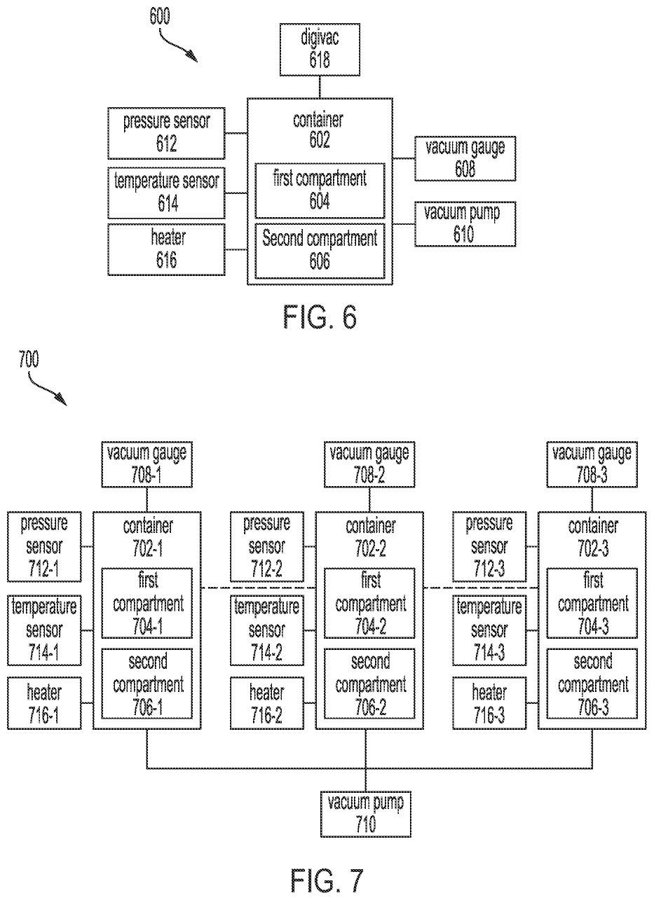

FIG. 7 illustrates a block diagram of multiple container apparatus 700 for infusing botanicals with terpene in accordance with an embodiment of the present invention. It represents an arrangement for the production of infused botanicals on a large scale. The multiple apparatus comprises containers 702-1 (having a first compartment 704-1 and a second compartment 706-1), 702-2 (having a first compartment 704-2 and a second compartment 706-2), and 702-3 (having a first compartment 704-3 and a second compartment 706-3). The container 702-1 is further connected to a vacuum gauge 708-1, a pressure sensor 712-1, a temperature sensor 714-1 and a heater 716-1. The container 702-2 is further connected to a vacuum gauge 708-2, a pressure sensor 712-2, a temperature sensor 714-2 and a heater 716-2. The container 702-3 is further connected to a vacuum gauge 708-3, a pressure sensor 712-3, a temperature sensor 714-3 and a heater 716-3. Each container of the multiple container apparatus is connected to a vacuum pump 710 via a common connection.

### Example: Preparation of an Infused Cannabis Flowers Under Low-Pressure Conditions

1. Weigh flower prior to test (Bach Size=227 g).
2. Prepare isolated terpenes, aldehydes, ketones, and esters at 7.5 ml on 227 g of weight of dry flower, either separately as isolated components or a mixture of isolated components.
3. Add terpenes, aldehydes, ketones, and esters to the terpene compartment of a container.
4. Place 100 micron steel mesh on top of tri-clamp filter plate and secure with compression ring.
5. Gently place flower into Tri-clamp spool on top of steel mesh.
6 Immediately seal the container by placing gasket on top section of spool and placing tri-clamp lid.
7. Place high pressure tri-clamps around spool and lid joint, and tighten bolts until completely sealed.
8. Close the ball valve attached to the container and the hose ball valve.
9. Turn on DigiVac. and turn on the vacuum.
10. Open hose ball valve. Slowly open ball valve attached to the container, to begin removing atmosphere with vacuum pump to a target vacuum of 16000 μm (micrometer Hg). This lowers the boiling point to 9.16° C. or 48.5 F. (Caution: open ball valve slowly as to not agitate flower).
11. Once vacuum gauge reads a target vacuum of 16000 μm, close ball valve on top of the container that was vacuumed out as well as the ball valve for the hose.
12. Set temperature on digital heating mat to 69 F (20.56° C.), if the container is at ideal target vacuum.
13. Once target temperature is achieved, allow to heat for 2 hours under vacuum.
14. At the 2 hour mark, shut off heat and let sit under vacuum for 1.5 additional hours.
15. Open the ball valve attached to the container very slowly to allow air to fill the container without agitating the flower. Close ball valve on the container and allow to sit for 30 minutes under normal atmosphere.
16. Remove high pressure tri-clamp and remove tri-clamp lid.
17 Immediately remove flower and place in an airtight terp-lock storage bags with humidity pack. The flower can be stored for longer periods by placing 0.5 ml-1 ml of terpene directly onto the humidity pack and placing in the storage bag.

The present invention has been described in terms of various examples. Many of these examples are directed at cannabis terpenes but can also include a multitude of volatile components found in botanicals, including Cannabis sativa. The result of the invention and its processes includes an infused botanical that has improved smell, flavor and shelf life. However the true scope of the invention is defined in the appended claims.

1. Infused cannabis flower, comprising:
dried cannabis flower having a maximum of 20% moisture content, an initial terpene profile, and an initial aldehyde profile;
the dried cannabis flower being infused with at least one isolated terpene selected from the group consisting of β-caryophyllene, bisabolol, carene, eucalyptol, humulene, limeonene, linalool, myrcene, neroidol, β-ocimene, pinene, terpineol, terpinolene or a combination thereof, to define an infused terpene profile; and
the dried cannabis flower being further infused with at least one isolated aldehyde selected from the group consisting of benzaldehyde, decanal, heptanal, octanal, hexanal, isobutyraldehyde, and combinations thereof, to define an infused aldehyde profile,
wherein the dried cannabis flower is infused with both terpenes and aldehydes to create the infused cannabis flower, and the initial terpene profile and the initial aldehyde profile have less total terpenes and total aldehydes than the infused terpene profile and the infused aldehyde profile of the infused cannabis flower, respectively.

2. The infused cannabis flower as set forth in claim 1, wherein the infused terpene profile including at least one terpene in a higher concentration than the concentration expressed in the initial terpene profile, so that the infused cannabis flower has a terpene concentration of at least 3.5% w/w.

3. The infused cannabis flower as set forth in claim 1, wherein the dried cannabis flower is further infused with aromatic molecules selected from the group consisting of esters, ketones, and combinations thereof.

4. The infused cannabis flower as set forth in claim 3, wherein the ketones are selected from the group consisting of 2-methyl-4-heptanone, 2-methyl-3-heptanone, methylisohexenyl, 1-chloroacetophenone, and combinations thereof.

5. The infused cannabis flower as set forth in claim 3, wherein the esters are selected from the group consisting of hexyl acetate and methyl anthranilate, linalyl acetate and combinations thereof.

6. The infused cannabis flower as set forth in claim 3, wherein the esters include tetrahydrocannabinolic acid esters.

7. The infused cannabis flower as set forth in claim 3, wherein the esters are tetrahydrocannabinolic acid esters.

8. The infused cannabis flower as set forth in claim 7, wherein the tetrahydrocannabinolic acid esters are selected from the group consisting of B-fenchyl-delta-9-tetrahydrocannabinolate, a-fenchyl-delta-9-tetrahydrocannabinolate, epi-bornyl-delta-9-tetrahydrocannabinolate; bornyl-delta-9-tetrahydrocannabinolate, a-terpenyl-delta-9-tetrahydrocannabinolate, 4-terpenyl-delta-9-tetrahydrocannabinolate; a-cadinyl-delta-9-tetrahydrocannabinolate, y-eudesmyl-delta-9-tetrahydrocannbinolate, and combinations thereof.

9. The infused cannabis flower as set forth in claim 3, wherein the ketones are selected from the group consisting essentially of 2-methyl-4-heptanone, 2-methyl-3-heptanone, methylisohexenyl, 1-chloroacetophenone, and combinations thereof.

10. The infused cannabis flower as set forth in claim 3, wherein the esters are selected from the group consisting essentially of hexyl acetate and methyl anthranilate, linalyl acetate and combinations thereof.

11. The infused cannabis flower as set forth in claim 3, wherein the esters include cannabigerol esters.

12. The infused cannabis flower as set forth in claim 11, wherein the cannabigerol esters are selected from the group consisting essentially of y-eudesmyl cannabigerolate and a-cadinyl cannabigerloate.

13. Infused cannabis flower, comprising:
dried cannabis flower having a shelf life and naturally occurring cannabinoids including acid forms of tetrahydrocannabinol and cannabidiol and at least one terpene selected from β-caryophyllene, bisabolol, carene, eucalyptol (cineole), humulene, limeonene, linalool, myrcene, neroidol, β-ocimene, pinene, terpineol, terpinolene or a combinations thereof; and
the dried cannabis flower being infused with additional terpenes selected from the group consisting of β-caryophyllene, bisabolol, carene, eucalyptol (cineole), humulene, limeonene, linalool, myrcene, neroidol, β-ocimene, pinene, terpineol, terpinolene or a combination thereof to yield terpene infused cannabis flower; and
the terpene infused cannabis flower being further infused with aldehydes selected from the group consisting of benzaldehyde, decanal, is heptanal, octanal, hexanal, isobutyraldehyde, and combinations thereof to yield a terpene and aldehyde infused cannabis flower; and
wherein the process of infusion occurs at a temperature of below 90° F. and pressure within the ranges of between 3160000 μm to 16000 μm.

14. The infused cannabis flower according to claim 13, wherein the naturally occurring terpenes define an initial terpene profile, which over time degrades at room temperature, the terpene infused cannabis flower regains the initial terpene profile to yield infused cannabis flower having an extended shelf life.

15. The infused cannabis flower according to claim 13, wherein the naturally occurring terpenes define an initial terpene profile, which over time degrades at room temperature, the additional terpenes cause the infused cannabis flower to exceed the initial terpene profile.

16. A process for infusing botanicals with terpenes comprising:
a) providing one or more terpenes in a first compartment;
b) providing one or more botanicals in a second compartment that is in fluid communication with the first compartment;
c) reducing pressure in both the first compartment and the second compartment to a vacuum reaching 16000 μm;
d) gently heating the first compartment under the vacuum to vaporize the one or more terpenes;
e) allowing the one or more terpene vapors to penetrate the one or more botanicals; and
f) collecting the infused botanicals.

17. The process according to claim 16, wherein the amount of the one or more terpenes is 1% to 15% w/w of the one or more botanicals.

18. The process according to claim 16, wherein the amount of the one or more terpenes is between 2.5 to 3.5% w/w of the one or more botanicals.

19. The process according to claim 16, wherein the second compartment has a temperature below that of the first compartment.

20. The process according to claim 16, wherein the one or more terpenes are selected from the group consisting of a β-caryophyllene, bisabolol, carene, eucalyptol (cineole), humulene, limeonene, linalool, myrcene, neroidol, β-ocimene, pinene, terpineol, terpinolene or a combination thereof.

21. The process according to claim 16, wherein heating the first compartment under vacuum at a negative pressure range from 3160000 μm to 16000 μm.

22. The process according to claim 16, wherein heating the first compartment under vacuum at a temperature range from 20° C.-55° C.

23. The process according to claim 16, wherein the time for allowing the terpene vapors to penetrate the botanicals is at least 1.5 hours.

24. The process according to claim 16, wherein collecting the infused botanicals is followed by recovering unused terpenes.

25. The process according to claim 16, further comprising infusing the infused botanical with at least one isolated aldehyde selected from the group consisting of benzaldehyde, decanal, heptanal, octanal, hexanal, isobutyraldehyde, and combinations thereof, to define an infused aldehyde profile.

26. The process according to claim 25, wherein the infused botanical is further infused with aromatic molecules selected from the group consisting of esters, ketones, and combinations thereof.

27. The process according to claim 26, wherein the ketones are selected from the group consisting of 2-methyl-4-heptanone, 2-methyl-3-heptanone, methyl isohexenyl, 1-chloroacetophenone, and combinations thereof.

28. The process according to claim 26, wherein the esters are selected from the group consisting of hexyl acetate and methyl anthranilate, linalyl acetate and combinations thereof.

29. Infused cannabis flower in a rolled cigarette, comprising:
dried cannabis flower rolled into a cigarette having rolling paper enclosing the dried cannabis flower, the dried cannabis flower having a maximum of 20% moisture content, an initial terpene profile, and an initial aldehyde profile;
the dried cannabis flower and rolling paper being infused with at least one isolated terpene selected from the group consisting of β-caryophyllene, bisabolol, carene, eucalyptol, humulene, limeonene, linalool, myrcene, neroidol, β-ocimene, pinene, terpineol, terpinolene or a combination thereof, to define an infused terpene profile; and
the dried cannabis flower and rolling paper being further infused with at least one isolated ester selected from the group consisting of hexyl acetate, methyl anthranilate, linalyl acetate and combinations thereof.

30. Infused cannabis flower, comprising:
dried cannabis flower having a maximum of 20% moisture content, an initial terpene profile, and an initial aldehyde profile;
the dried cannabis flower being infused with at least one isolated terpene selected from the group consisting of β-caryophyllene, bisabolol, carene, eucalyptol, humulene, limeonene, linalool, myrcene, neroidol, β-ocimene, pinene, terpineol, terpinolene or a combination thereof, to define an infused terpene profile; and
the dried cannabis flower being further infused with at least one isolated ester selected from the group consisting of hexyl acetate, methyl anthranilate, linalyl acetate and combinations thereof.

31. A cellulosic product package, comprising:
cellulosic packaging material infused with at least one isolated terpene selected from the group consisting of β-caryophyllene, bisabolol, carene, eucalyptol, humulene, limeonene, linalool, myrcene, neroidol, β-ocimene, pinene, terpineol, terpinolene or a combination thereof, to define an infused terpene profile; and
the cellulosic packaging material being infused with at least one isolated ester selected from the group consisting of hexyl acetate, methyl anthranilate, linalyl acetate and combinations thereof.
